(19) United States
(12) Patent Application Publication
(10) Pub. No.: US 2026/0128169 A1
(43) Pub. Date: May 7, 2026

(54) TRANSFERABLE AND INTERPRETABLE TREATMENT EFFECTIVENESS PREDICTION FOR OVARIAN CANCER VIA MULTIMODAL DEEP LEARNING

(71) Applicant: University of Southern California, Los Angeles, CA (US)

(72) Inventors: Yan Liu, Los Angeles, CA (US); Joseph Carlson, Los Angeles, CA (US); Emily Nguyen, Los Angeles, CA (US)

(21) Appl. No.: 19/381,256

(22) Filed: Nov. 6, 2025

Related U.S. Application Data
(60) Provisional application No. 63/717,422, filed on Nov. 7, 2024.

(51) Int. Cl. G16H 50/20

(52) U.S. Cl. CPC G16H 50/20

(57) ABSTRACT

A multimodal deep learning framework which is used to determine the likelihood of a particular treatment method effectively treating a patient with ovarian/kidney cancer with the goal of increasing patient survival. The framework takes into account not only large histopathology images (whole slide images), but also clinical variables to increase the scope of the data. The results demonstrate that the proposed models achieve high prediction accuracy and interpretability and can also be transferred to other cancer datasets without significant loss of performance. One of the key innovations here is the combination of pathology and clinical variables in a deep learning model to provide recommendations in therapy areas with limited information.

### CROSS REFERENCE TO RELATED APPLICATION

This application claims priority to U.S. Provisional Patent Application No. 63/717,422, filed Nov. 7, 2024, entitled “TRANSFERABLE AND INTERPRETABLE TREATMENT EFFECTIVENESS PREDICTION FOR OVARIAN CANCER VIA MULTIMODAL DEEP LEARNING,” the disclosure of which is expressly incorporated herein by reference.

### BACKGROUND

In the United States, approximately 22,000 people are diagnosed with ovarian cancer each year, and approximately 14,000 people will die of this disease. A problem that leads to such a high death rate is that current clinical practice follows a series of prescribed chemotherapeutic regimens even though a significant number of patients will not respond to these standard drugs. If resistance could be identified before treatment, patients could be offered alternative, potentially more appropriate, drugs. In the context of ovarian cancer, deep learning models have been applied to tasks such as tumor segmentation and classification, survival prediction, and treatment effectiveness prediction. Some deep learning models attempt to discover individualized disease factors to improve treatment decisions.

Despite promising results, there are still several challenges associated with applying deep learning models to ovarian cancer research. One major challenge is the need for large and diverse datasets to train these models. The digitization of patient tissue samples enables global distribution of the data and high-throughput screening of patients for diagnostic and research application settings. Specifically, digital whole slide images (WSIs) are being increasingly used for both ovarian cancer research, as well a routine diagnostics. While WSIs have the potential to develop the workflow of pathologists further, the collection of WSI images requires significant technical expertise, resources, and infrastructure, and may not be feasible in all settings. Furthermore, a lack of model interpretability can lead to the failure to understand the biological mechanisms and ensure ethical considerations.

Thus, there is a critical need for innovations that can assist in the selection of chemotherapy for these patients.

### SUMMARY

Ovarian cancer, a potentially life-threatening disease, is often difficult to treat. There is a critical need for innovations that can assist in improved therapy selection. Although deep learning models show promising results, they are employed as a “black-box” and require enormous amounts of data. Therefore, the present disclosure describes methods and systems for the transferable and interpretable prediction of treatment effectiveness for ovarian cancer patients. A multimodal deep learning framework is described that accounts for not only large histopathology images but also clinical variables to increase the scope of the data. The results demonstrate that the models achieve high prediction accuracy and interpretability. Further, the systems and methods can be transferred to other cancer datasets without significant performance loss.

Described herein is a method for predicting treatment effectiveness of a disease condition. The method may include receiving histopathology images of tissue; using a trained transferrable feature embedder to derive embeddings from the histopathology images; combining the embeddings with predetermined clinical variable features associated with a predictive importance relative to the disease condition; classifying the combined embeddings and the predetermined clinical variable features for predicting the treatment effectiveness; and outputting a visualization of feature interactions and a score. A corresponding system is also described herein.

In accordance with another aspect, an interpretable and transferable multimodal deep learning framework for assessing whole slide images (WSIs) is disclosed. The framework may operate on a computing device that executes: a data pre-processing component that receives the WSIs; a multimodal deep learning component includes a feature embedder that derives embeddings from the WSIs and combines the embeddings with predetermined clinical variable features associated with a predictive importance relative to the disease condition; and an interpretable prediction component that classifies and predicts a treatment effectiveness from the combined embeddings and the predetermined clinical variable features. The framework outputs a visualization of feature interactions and a score, and wherein interpretable insights associated with the classifying.

This summary is provided to introduce a selection of concepts in a simplified form that is further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative implementations, is better understood when read in conjunction with the appended drawings. To illustrate the implementations, there are shown in the drawings example constructions; however, the implementations are not limited to the specific methods and instrumentalities disclosed. In the drawings:

FIG. 1 illustrates an overview of an interpretable and transferable multimodal deep learning framework in accordance with aspects of the present disclosure;

FIGS. 2A, 2B, 2C and 2D illustrate shape functions for four selected clinical variable features;

FIGS. 3 and 4 illustrate selected whole slide images (WSIs), heatmaps, and their high attention patches (red) from Bevacizumab effective patients; and

FIG. 5 is an example computing device in accordance with aspects of the present disclosure.

### DETAILED DESCRIPTION

### Introduction

The present disclosure describes systems and methods for the interpretable and transferable prediction of treatment effectiveness for ovarian cancer patients. A multimodal deep learning framework considers both large histopathology images and clinical variables. In addition to providing treatment effectiveness prediction, the present disclosure improves interpretability by providing insights into the factors that influence treatment effectiveness in ovarian cancer patients. The disclosed model may also be applied to kidney cancer research, thus demonstrating that the disclosed model is transferable to different cancers at a feature-level given digital whole slide images (WSIs) and clinical variables. The interpretable and transferable approach is adapted to assist a medical practitioner's selection of chemotherapy in a trustworthy and data-efficient fashion.

As will be described, the present disclosure provides a multimodal deep learning framework, interpretable prediction, and feature-level transferring to other cancers (e.g., kidney cancer). The multimodal deep learning framework, which utilizes not only large histopathology images, but also clinical variables can predict treatment effectiveness using histopathology images, without requiring any pathologist-provided locally annotated regions, and clinical variables. The described methods and systems can interpret the predictions via an interpretability method that provides visualizations of feature interactions and patch attention scores. Insights into predictions of the classifier, and thus, the factors that influence treatment effectiveness in ovarian cancer patients are provided. The interpretable prediction helps inform personalized treatment decisions. The transferable nature of the present disclosure enables the methods and systems to perform prediction on other cancers, such as kidney cancer in a data-efficient manner.

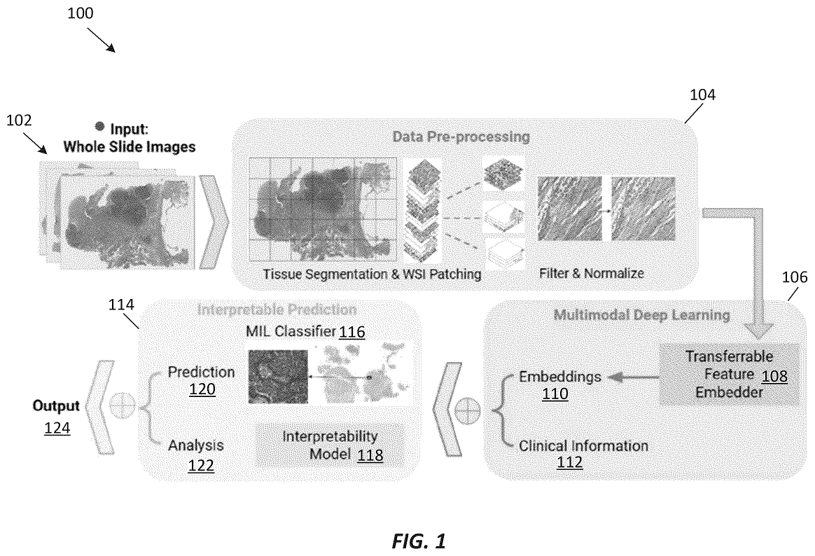

With reference to FIG. 1, there is illustrated an overview of an interpretable and transferable multimodal deep learning framework 100. The framework receives digital whole slide images (WSIs) 102 as an input. The WSIs 102 are provided to a data pre-processing component 104 that may use an OpenSlide library, which is a software tool for interfacing with WSIs. Other software tools may be used to perform the following. In the data pre-processing component 104, each WSI is down sampled, e.g., three times, and then is made grayscale. An edge detection algorithm may be applied to the grayscale WSI to extract edges. These steps advantageously reduce the computational resources required in subsequent model training. For example, the WSI is tiled into 64×64 pixels per tile. Tiling is performed since the WSIs may be too large to be input directly into a deep learning model. The tiles are filtered by image entropy greater than, e.g., five which removes images with no tissue in them. This process is applied to the WSIs without any pathologist-provided locally annotated regions.

The output of the data pre-processing component 104 is provided to a multimodal deep learning component 106. Multimodal deep learning makes use of all available information from different sources (i.e., multiple modalities) to improve deep learning models' performance. Multimodal deep learning intuitively mimics real-world diagnostic procedures by employing unique feature observations during medical screening and diagnostics. Employing a multimodal deep learning approach has been shown to improve prediction accuracy.

A feature embedder 108, using, for example, a ResNet model, may be trained using the WSIs. The embedding for both instance-level and bag-level are learned simultaneously. The learned embeddings 110 are output from the feature embedder 108 and are concatenated with clinical information 112 (e.g., tabular clinical variables, described below) to linearly reduce the data. The clinical information 112 may be clinical variable features selected based on their expected predictive importance. The categorical features may be one-hot encoded to transform the data into numbers to ensure that the algorithm does not interpret higher numbers as more important. Ordinal features may be label encoded and the data normalized using a z-score.

The output of the multimodal deep learning component 106 is provided as an interpretable prediction 114. Here, the combined features are input into a MIL classifier 116 and an interoperability model 118 to provide a prediction 120 and analysis 122 as an output 124 of the framework 100. Interpretable insights using, e.g., Sparse Interaction Additive Networks (SIAN), which an effective selection algorithm inspired by feature interaction detection that identifies the feature combinations in large-scale tabular datasets. The interaction detection procedure in SIAN is used in order to select relevant features from the tabular medical records to build the interpretable model 118 that utilizes the feature interactions selected by this procedure.

Below are additional details, specific examples, and experimental results of the WSI datasets, pre-processing, multimodal deep learning and interpretable prediction performed by the framework 100 of FIG. 1.

### Model Specifics and Training

Two example datasets were provided to the framework 100 as the input 102 to determine the effectiveness of the predictions provided thereby. One dataset utilized was an Ovarian Bevacizumab Dataset. This dataset consists of hematoxylin and eosin (H&E) WSIs. In total, there are 288 de-identified H&E stained WSIs with 162 being effective and 126 being invalid. The WSIs were acquired with a Leica AT2 digital scanner with a 20× objective lens. The resolution of WSIs is 54342×41048 in pixels on average. Clinical information of epithelial ovarian cancer (EOC) patients and peritoneal serous papillary carcinoma (PSPC) patients are also provided. The clinical variables of EOC and PSPC patients are collected from 78 patients at the Tri-Service General Hospital and the National Defense Medical Center, Taipei, Taiwan. The clinical variables 112 are composed of age, diagnosis, FIGO stage, operation type, method for avastin use, days from the operation date to the starting date for use of avastin, and BMI.

The second dataset is a Kidney Dataset. This dataset consists of WSIs from ten patients with clear cell renal cell carcinoma (ccRCC), a subtype of kidney cancer. The stained slides are scanned at an objective of 20× object magnification on an Axioscan Zeiss digital scanner. In total, there are 10 WSIs collected from 10 patients. The WSIs are around 100,000×100,000 pixels in size. The clinical variables are also provided and included gender, age at surgery, disease-free months, fuhrman nuclear grade, ISUP nuclear grade, tumor stage, tumor size, node status, necrosis, leibovich score (Fuhrman), leibovich score (ISUP). The number of patients with and without cancer recurrence in the next five years is equal.

Using the ResNet model, the feature embedder was trained from scratch using all WSIs. The learned bag embedding is of dimension (number of instances in a bag, 512). The bag embeddings were concatenated with 22 tabular clinical variables and linearly reduced the data to 20 components using PCA. The resulting vectors were input into the MIL classifier model and trained using Adam optimization, a learning rate of 1e-5, and weight decay of 1e-5. The model was trained over 100 epochs with a batch size of 128 using the Cross Entropy Loss function. The ovarian Bevacizumab dataset was split into 80% training 20% test sets. The kidney dataset was split into 50% training 50% test sets.

### Interpretability

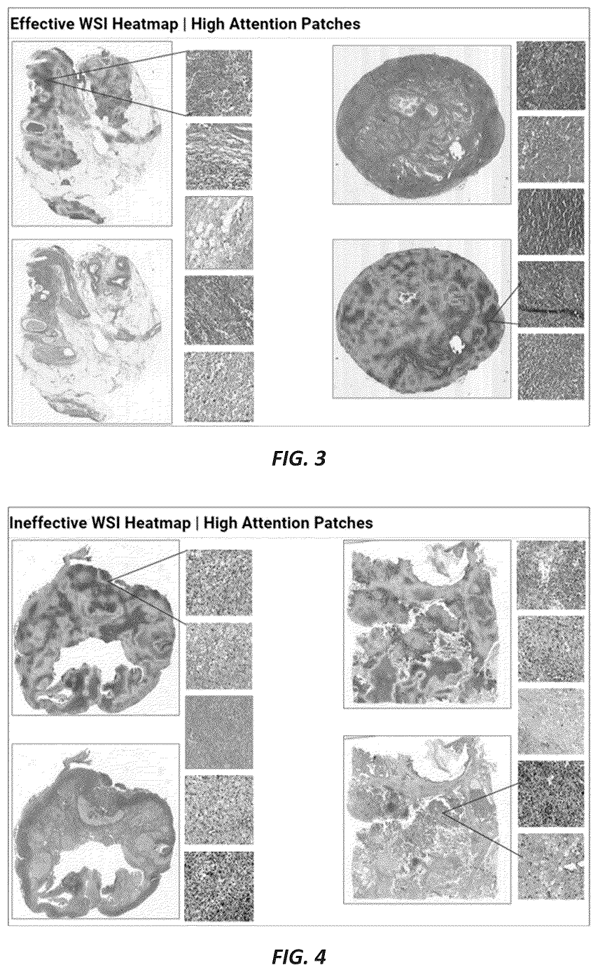

Deep neural networks that are trained as black box predictors do not give insights into the reasoning behind their recommendations. Herein, the interpretability techniques of feature interaction detection and additive modeling of the framework 100 provide improvements as they are used to provide interpretable insights from the machine learning models 118. In addition, the CLAM attention-based learning framework is used to generate attention heatmaps of the WSIs (shown in FIGS. 3 and 4, described below). The CLAM framework first extracts WSI patches. These patches are encoded using a pre-trained CNN, ranked based on their relative importance to the slide-level prediction, and assigned attention scores based on their rank. The process of creating an attention heatmap then involves converting model attention scores to percentiles and mapping them spatially to the original slide. Visualizing attention scores as a heatmap captures the relative contribution and importance of each patch to the model's predictions. This visualization can also aid in identifying key diagnostic features.

### Metrics

The accuracy of the framework 100 is by measuring the area under the curve (AUC) metric, which evaluates the area under the Receiver Operating Characteristic (ROC) curve between the True Positive Rate (TPR) and the False Positive Rate (FPR). Accuracy is calculated as the ratio of the number of correctly predicted WSIs to the total number of WSI in the dataset. On the kidney dataset, we perform across 3-fold cross-validation and report the average of the scores.

### Results

To show the effectiveness of the framework 100, the model is compared to two baselines: 1) image-only baseline which performs treatment effectiveness prediction with WSIs only; and 2) clinic-only baseline which performs treatment effectiveness prediction with tabular clinical variables only. The AUC and accuracy results for the disclosed framework 100 and baseline methods are shown in Table 1. In Table 1, DSMIL refers to dual-stream multi-instance learning.

TABLE 1
Comparison to baseline models

| Methods | AUC | Accuracy |
|---|---|---|
| DSMIL | 0.780 | 0.780 |
| Clinic-only | 0.767 | 0.730 |
| Multimodal (this | 0.796 | 0.783 |
| disclosure) | | |

The decision to administer therapy drugs is a complex problem that does not rely solely on WSIs or clinical variables alone. Clinicians consider other factors, such as patient factors and treatment risks. As described herein, the framework 100 addresses these limitations. These results demonstrate that the multimodal deep learning component 106 effectively combines the available WSIs and clinical variables 112 to provide better performance compared to a single data source.

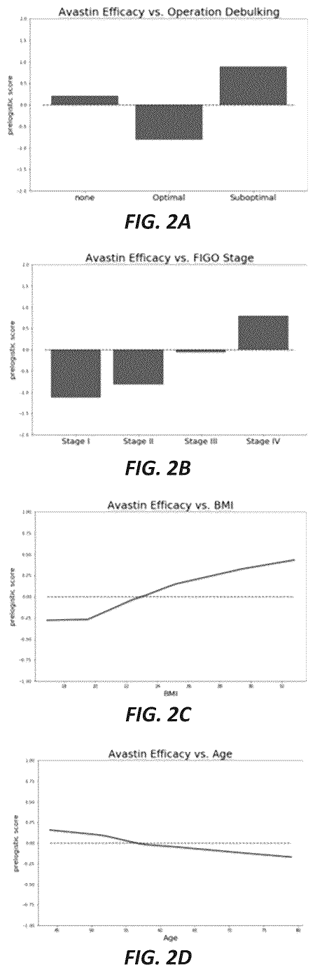

The resulting interpretably model 118 can be effectively visualized using shape functions in FIGS. 2A-2D generated by the SIAN model. In FIG. 2A, suboptimal operation debulking and no debulking are expected to correspond to high risk of ineffective treatment, while optimal debulking corresponds to low risk of ineffective treatment because there is more residual tumor remaining after the operation. In FIG. 2B, that risk of ineffective treatment is shown as increasing with FIGO stage severity. This observation aligns with clinical expertise since earlier stages of cancer are more likely to be treated effectively than later stages of cancer. The feature interaction between patient BMI and risk of avastin ineffectiveness is shown in FIG. 2C. The plot suggests that a lower BMI corresponds to lower risk of ineffective treatment, and that the risk increases as patient BMI increases. In FIG. 2D, the feature interaction suggests that an older-aged patient may have a moderately lower risk of ineffective treatment than younger-aged patients.

In clinical settings, it is important to be able to communicate high-stakes decisions not only to the doctor but also to the patient. When external algorithms are unable to explain their decisions, medical professionals often choose to ignore black-box decisions instead of endangering their patient's ability to fully understand the risks and benefits of a procedure. As a result, the framework 100 of present disclosure presents visualizations of feature interactions. The framework demonstrates that these interactions align with clinical expertise, ensuring that predictions are accurate and reliable.

FIGS. 3 and 4 are example visualizations of an output of the adapted CLAM framework. Areas of high attention patches (red) often occur in the tumor regions of the WSI, while low attention patches (blue) often occur in benign fibrous tissue and non-tumor regions. The high attention patches provide additional interpretability, allowing clinicians to observe finer details within the WSI tissue such as necrosis, growth patterns, and cell nuclei.

As noted above, the implementations herein provide for feature-level transferring to other cancers, such as kidney cancer. Various data preprocessing techniques (e.g. one-hot encoding, normalization) may be used to extract machine-learning ready features from the clinical variables. In particular, the transferable feature embedder trained on the ovarian Bevacizumab dataset may be employed to extract embedders for the WSIs in the kidney dataset. To demonstrate the effectiveness of the feature-level transferring, the performance to the baseline where the feature embedder is not trained on the Bevacizumab dataset is compared below. The AUC and accuracy results for the disclosed and the baseline methods are shown in Table 2.

TABLE 2
Feature-level transferring to kidney dataset.

| Methods | AUC | Accuracy |
|---|---|---|
| w/o Bevacizumab | 0.400 | 0.640 |
| w Bevacizumab (this | 0.582 | 0.720 |
| disclosure) | | |

The effectiveness of the feature-level transferring to the kidney cancer dataset is shown. In particular, training the feature embedder on the ovarian Bevacizumab dataset achieves better performance (AUC 0.582) than the baseline (AUC 0.400). These results suggest potential for transferring learning applications between different cancer datasets.

### CONCLUSIONS

Thus, a transferable and interpretable treatment effectiveness prediction for ovarian cancer is described herein. In particular, the multimodal deep learning framework 100 performs treatment effectiveness prediction based on both large histopathology images and tabular clinical information. In addition to predicting treatment effectiveness, the predictions are interpreted by an-art interpretability model. To improve data-efficiency, the feature-level transferring from ovarian cancer to kidney cancer is also described herein.

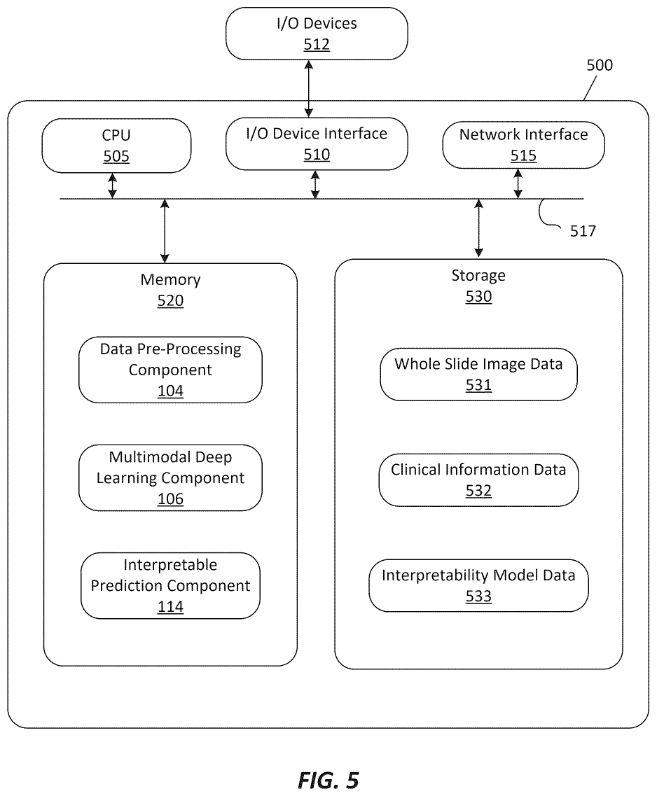

FIG. 5 illustrates examples of computers 500 that may include the kinds of software programs, data stores, and hardware that can implement event message processing, context determination, notification generation, and content delivery, as described above according to certain embodiments. As shown, the computing system 500 includes, without limitation, a central processing unit (CPU) 505, a network interface 515, a memory 520, and storage 530, each connected to a bus 517. The computing system 500 may also include an i/o device interface 510 connecting i/o devices 512 (e.g., keyboard, display and mouse devices) to the computing system 500. Further, the computing elements shown in computing system 500 may correspond to a physical computing system (e.g., a system in a data center) or may be a virtual computing instance executing within a computing cloud.

The CPU 505 retrieves and executes programming instructions stored in the memory 520 as well as stored in the storage 530. The bus 517 is used to transmit programming instructions and application data between the CPU 505, I/O device interface 510, storage 530, network interface 515, and memory 520. Note, CPU 505 is included to be representative of a single CPU, multiple CPUs, a single CPU having multiple processing cores, and the like, and the memory 520 is generally included to be representative of a random access memory. The storage 530 may be a disk drive or flash storage device. Although shown as a single unit, the storage 530 may be a combination of fixed and/or removable storage devices, such as fixed disc drives, removable memory cards, optical storage, network attached storage (NAS), or a storage area-network (SAN).

Illustratively, the memory 520 includes one or more of the data pre-processing component 104, multimodal deep leaning component 106, and/or interpretable prediction component 114, all of which are discussed in greater detail above. Further, storage 530 includes one or more of, whole slide image (WSIs) data 531, clinical information data 532, and/or interpretability model data 535, all of which are also discussed in greater detail above.

It should be understood that the various techniques described herein may be implemented in connection with hardware components or software components or, where appropriate, with a combination of both. Illustrative types of hardware components that can be used include field-programmable gate arrays (FPGAS), application-specific integrated circuits (ASICS), application-specific standard products (ASSPS), system-on-a-chip systems (SOCS), complex programmable logic devices (CPLDS), etc. The methods and apparatus of the presently disclosed subject matter, or certain aspects or portions thereof, may take the form of program code (i.e., instructions) embodied in non-transitory, tangible media, such as removeable drives (floppy diskettes, CD-ROMS), hard drives, including such on cloud-based environments, or any other machine-readable storage medium where, when the program code is loaded into and executed by a machine, such as a computer, the machine becomes an apparatus for practicing the presently disclosed subject matter.

Although certain implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A method for predicting treatment effectiveness of a disease condition, comprising:
receiving histopathology images of tissue;
using a trained transferable feature embedder to derive embeddings from the histopathology images;
combining the embeddings with predetermined clinical variable features associated with a predictive importance relative to the disease condition;
classifying the combined embeddings and the predetermined clinical variable features for predicting the treatment effectiveness; and
outputting a visualization of feature interactions and a score.

2. The method of claim 1, further comprising preprocessing the histopathology images to extract edges from within the histopathology images.

3. The method of claim 2, further comprising down sampling the histopathology images and converting the down sampled histopathology images to grayscale and determining the edges from the grayscale.

4. The method of claim 3, further comprising:
tiling the grayscale images for input to the transferable feature embedder; and
filtering the tiled images to remove images that do not contain tissue.

5. The method of claim 1, wherein the histopathology images are whole slide images (WSIs).

6. The method of claim 1, wherein the predetermined clinical variable features are associated with at least one of age, diagnosis, FIGO stage, operation type, method for avastin use, days from an operation date to a starting date for use of avastin, and BMI.

7. The method of claim 1, wherein the visualization is presented as a heatmap that indicates a relevance of regions in the histopathology images to the predicting.

8. The method of claim 1, further comprising providing interpretable insights associated with the classifying.

9. A system for predicting treatment effectiveness of a disease condition, comprising:
a storage device;
a memory; and
a processor,
wherein the processor executes:
a data pre-processing component that receives histopathology images of tissue;
a multimodal deep learning component that uses a trained transferable feature embedder to derive embeddings from the histopathology images and combines the embeddings with predetermined clinical variable features associated with a predictive importance relative to the disease condition; and
an interpretable prediction component that classifies the combined embeddings and the predetermined clinical variable features for predicting the treatment effectiveness and outputs a visualization of feature interactions and a score.

10. The system of claim 9, wherein the data pre-processing component pre-processes the histopathology images to extract edges from within the histopathology images.

11. The system of claim 10, wherein the pre-processing component down samples the histopathology images and converts the down sampled histopathology images to grayscale and determining the edges from the grayscale.

12. The system of claim 11, wherein the pre-processing component tiles the grayscale images for input to the transferrable feature embedder, and wherein the pre-processing component filters the tiled images to remove images that do not contain tissue.

13. The system of claim 9, wherein the histopathology images are whole slide images (WSIs).

14. The system of claim 9, wherein the predetermined clinical variable features are associated with at least one of age, diagnosis, FIGO stage, operation type, method for avastin use, days from an operation date to a starting date for use of avastin, and BMI.

15. The system of claim 9, wherein the visualization is presented as a heatmap that indicates a relevance of regions in the histopathology images to the predicting.

16. The system of claim 9, wherein the interpretable prediction component provides interpretable insights associated with the classifying.

17. A computer-readable medium containing program instructions for causing a computer to perform a method for predicting treatment effectiveness of a disease condition, comprising:
receiving histopathology images of tissue;
using a trained transferable feature embedder to derive embeddings from the histopathology images;
combining the embeddings with predetermined clinical variable features associated with a predictive importance relative to the disease condition;
classifying the combined embeddings and the predetermined clinical variable features for predicting the treatment effectiveness; and
outputting a visualization of feature interactions and a score.

18. The computer-readable medium of claim 17, further comprising instructions for down sampling the histopathology images and converting the down sampled histopathology images to grayscale and determining edges from the grayscale.

19. The computer-readable medium of claim 17, wherein the predetermined clinical variable features are associated with at least one of age, diagnosis, FIGO stage, operation type, method for avastin use, days from an operation date to a starting date for use of avastin, and BMI.

20. The computer-readable medium of claim 17, wherein the visualization is presented as a heatmap that indicates a relevance of regions in the histopathology images to the predicting.

21. The computer-readable medium of claim 17, further comprising instructions for providing interpretable insights associated with the classifying.
